(19) United States
(12) Patent Application Publication
(10) Pub. No.: US 2023/0218267 A1
(43) Pub. Date: Jul. 13, 2023

(54) DRIVE SHAFT AND IMAGE DIAGNOSIS CATHETER

(71) Applicant: TERUMO KABUSHIKI KAISHA, Tokyo (JP)

(72) Inventor: Kenta SUZUKI, Fujinomiya (JP)

(73) Assignee: TERUMO KABUSHIKI KAISHA, Tokyo (JP)

(21) Appl. No.: 18/184,162

(22) Filed: Mar. 15, 2023

Related U.S. Application Data
(63) Continuation of application No. PCT/JP2021/035186, filed on Sep. 24, 2021.

(30) Foreign Application Priority Data
Sep. 29, 2020 (JP) 2020-163874

(51) Int. Cl. A61B 8/12; A61M 25/00

(52) U.S. Cl. CPC A61B 8/12; A61M 25/005; A61B 8/445

(57) ABSTRACT

A drive shaft for constituting an imaging core of an image diagnosis catheter includes: a coil shaft having a distal end fixed to a proximal end of a housing that accommodates a signal transmitter and receiver; and a shaft member fixed to a proximal end of the coil shaft and having higher torsional stiffness than torsional stiffness of the coil shaft. An image diagnosis catheter includes: an imaging core including the drive shaft, the housing fixed to the distal end of the coil shaft, and the signal transmitter and receiver accommodated in the housing; and a sheath into which the imaging core is inserted.

### CROSS-REFERENCES TO RELATED APPLICATIONS

This application is a continuation of International Patent Application No. PCT/JP2021/035186 filed on Sep. 24, 2021, which claims priority to Japanese Patent Application No. 2020-163874 filed on Sep. 29, 2020, the entire content of both of which is incorporated herein by reference.

### TECHNOLOGICAL FIELD

The present disclosure generally relates to a drive shaft and an image diagnosis catheter.

### BACKGROUND DISCUSSION

There is known an image diagnosis catheter that enables diagnosis by transmitting a signal that is an ultrasound wave and/or light in a body cavity of a living body and receiving a reflected wave thereof to image the surface and the inside of a lesion portion. An example is described in Japanese Patent Application Publication No. 2006-198425 (JP 2006-198425 A). The image diagnosis catheter is configured to generate an image by retracting an imaging core having a signal transmitter and receiver while rotating the imaging core at a predetermined rotational speed in a sheath.

The imaging core includes a housing accommodating the signal transmitter and receiver and a drive shaft fixed to a proximal end of the housing, and is rotationally driven by an external device. The drive shaft is usually formed using a coil shaft made of multi-layer multi-wire coils as described in Japanese Patent Application Publication No. 2006-198425 extending over the entire length of the drive shaft.

### SUMMARY

The imaging core described above normally enables image generation by repeating transmission and reception while rotating at a constant rotational speed of about 1000 to 10000 rpm. However, when contact between the sheath and the imaging core occurs as the sheath is bent due to a flexure or a lesion of the biological lumen, the drive shaft sometimes resonates due to the contact. When the resonance occurs, an actual rotational speed of the signal transmitter and receiver deviates from a theoretical value, and image distortion called non-uniformed rotational distortion (NURD) occurs.

The drive shaft and image diagnosis catheter disclosed here are capable of suppressing occurrence of NURD.

An image core drive shaft that is to be used in an image diagnosis catheter comprises: a coil shaft having a distal end configured to be fixed to a proximal end of a housing that accommodates a signal transmitter and receiver, with the coil shaft possessing a proximal end and a distal end, and the coil shaft including at least one wire that is wound into a coil, and a shaft member fixed to the proximal end of the coil shaft and having higher torsional stiffness than torsional stiffness of the coil shaft.

As an embodiment of the present disclosure, an axial length of the coil shaft is 250 mm or more and 1000 mm or less.

As an embodiment of the present disclosure, an axial length of the shaft member is 200 mm or more and 1750 mm or less.

As an embodiment of the present disclosure, the shaft member is a tube having a notch.

As an embodiment of the present disclosure, the notch has a non-spiral shape.

As an embodiment of the present disclosure, the notch includes a plurality of slits extending along a circumferential direction.

As an embodiment of the present disclosure, the shaft member includes a main portion in which a plurality of slits each having a predetermined width and arranged in the circumferential direction are disposed in a predetermined pattern where the slits are arranged side by side at a predetermined pitch in an axial direction.

As an embodiment of the present disclosure, the shaft member includes a weakened portion having lower torsional strength than torsional strength of each of the main portion and the coil shaft.

As an embodiment of the present disclosure, a plurality of slits are disposed in the weakened portion in a pattern identical to the pattern of the main portion except that a predetermined width and/or a predetermined pitch are/is smaller than the predetermined width and/or the predetermined pitch of the main portion.

As an embodiment of the present disclosure, the weakened portion is located closer to a proximal end side than the main portion.

As an embodiment of the present disclosure, the predetermined pattern is a pattern in which pairs of slits, each of the pairs of slits facing each other in a radial direction and each having the predetermined width and extending along the circumferential direction, are disposed to be arranged side by side at the predetermined pitch in the axial direction while rotating by a predetermined angle.

An image diagnosis catheter according to another aspect of the present disclosure includes: an imaging core including the drive shaft as the first aspect of the present disclosure, the housing fixed to the distal end of the coil shaft, and the signal transmitter and receiver accommodated in the housing; and a sheath into which the imaging core is inserted.

According to the present disclosure, it is possible to provide the drive shaft and the image diagnosis catheter which are capable of suppressing the occurrence of NURD.

According to another aspect, a method comprises positioning an imaging core, located in a lumen of a surrounding sheath, inside a body cavity in a living body. The imaging core comprises a rotatable and axially movable drive shaft, with the imaging core also comprising a housing in which is located a signal transmitter and receiver. The drive shaft is comprised of a coil shaft fixed to a distal end of a shaft member so that the coil shaft and the shaft member rotate and axially move together. The coil shaft has a distal end that is fixed to the housing so that rotation and axial movement of the drive shaft results in rotation and axial movement of the housing as well as the signal transmitter and receiver accommodated in the housing. The coil shaft includes at least one wire that is wound into a coil, and the shaft member has a higher torsional stiffness than a torsional stiffness of the coil shaft. The method additionally comprises retracting the imaging core in the lumen of the surrounding sheath and rotating the imaging core in the lumen of the surrounding sheath while the signal transmitter and receiver transmits and receives signals.

### BRIEF DESCRIPTION OF THE DRAWINGS

FIG. 1 is a plan view illustrating a state in which an external device is connected to an image diagnosis catheter as one embodiment.

FIG. 2A is a side view illustrating the image diagnosis catheter illustrated in FIG. 1 in a state before a pull-back operation.

FIG. 2B is a side view illustrating the image diagnosis catheter illustrated in FIG. 1 in a state after the pull-back operation.

FIG. 3 is a cross-sectional view illustrating a distal end of the image diagnosis catheter illustrated in FIG. 1.

FIG. 4 is a cross-sectional view illustrating a proximal end of the image diagnosis catheter illustrated in FIG. 1.

FIG. 5 is a side view of a drive shaft illustrated in FIG. 1.

FIG. 6 is a side view of a shaft member illustrated in FIG. 5.

FIG. 7 is a cross-sectional view taken along the section line VII-VII in FIG. 6.

FIG. 8 is a cross-sectional view taken along the section line VIII-VIII in FIG. 6.

### DETAILED DESCRIPTION

Hereinafter, embodiments of a drive shaft and an image diagnosis catheter representing examples of the new drive shaft and image diagnosis catheter disclosed here will be described in detail with reference to the drawings.

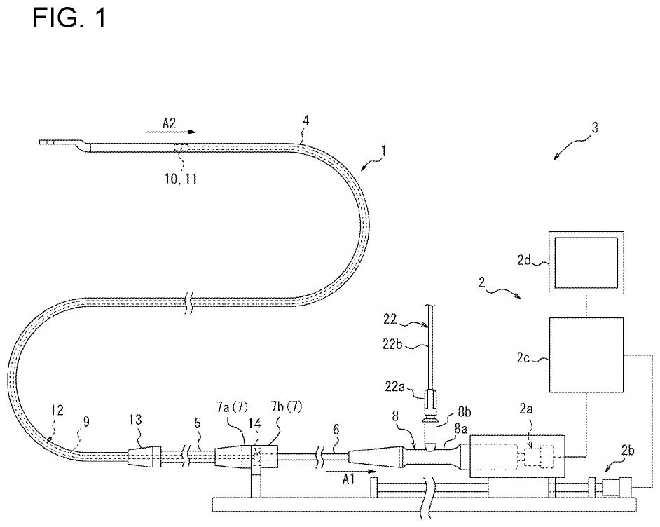

An image diagnosis catheter 1 according to the present embodiment is a dual type that uses both an intravascular ultrasound (IVUS) diagnosis method and an optical coherence tomography (OCT) diagnosis method. In the dual-type image diagnosis catheter 1, there are three types of modes including a mode of acquiring a tomographic image only by IVUS, a mode of acquiring a tomographic image only by OCT, and a mode of acquiring a tomographic image by both IVUS and OCT, and these modes can be used in a switched manner. As illustrated in FIG. 1, the image diagnosis catheter 1 is connected to an external device 2 and driven. The image diagnosis catheter 1 and the external device 2 constitute an image diagnosis apparatus 3.

As illustrated in FIGS. 1 to 4, the image diagnosis catheter 1 includes: a sheath 4 to be inserted into a body cavity such as a vessel (a blood vessel such as a coronary artery) of a living body; an outer tube 5 connected to a proximal end of the sheath 4; an inner tube 6 to be inserted into the outer tube 5 so as to be movable forward and backward; a unit connector 7 which is connected to a proximal end of the outer tube 5, holds the inner tube 6 so as to be movable forward and backward, and can release the holding of the inner tube 6; and a hub 8 connected to a proximal end of the inner tube 6. The image diagnosis catheter 1 further includes an imaging core 12 including a drive shaft 9 (imaging core drive shaft), a housing 10 fixed to a distal end of the drive shaft 9, and a signal transmitter and receiver 11 that is accommodated in the housing 10 and transmits and receives a signal that is an ultrasound wave and/or light. The imaging core 12 is inserted into and positioned in the sheath 4, the outer tube 5, and the inner tube 6, and can move forward and backward in an axial direction integrally with the inner tube 6 with respect to the sheath 4 and the outer tube 5.

In the present specification, the distal end means an end on a side of the image diagnosis catheter 1 to be inserted into a body cavity, the proximal end means an end on a side of the image diagnosis catheter 1 to be held outside the body cavity, the axial direction means a direction along a central axis O of the drive shaft 9 (that is, an extending direction of the drive shaft 9), a radial direction means a direction along a straight line orthogonal to the central axis O, and a circumferential direction means a direction around the central axis O.

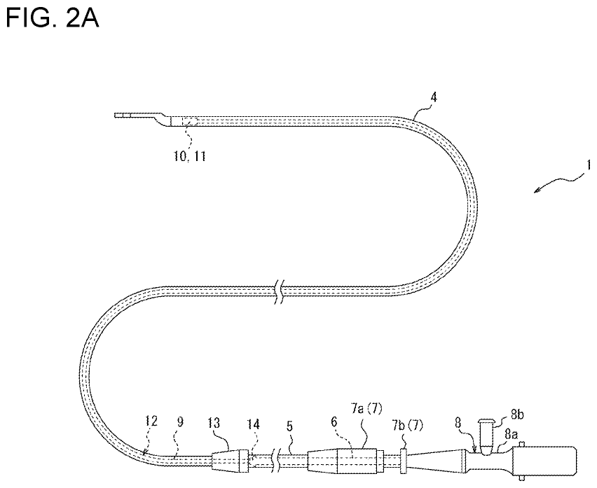

As illustrated in FIG. 2A, the drive shaft 9 extends to the inside of the hub 8 through the sheath 4, the outer tube 5, and the inner tube 6. The hub 8, the inner tube 6, the drive shaft 9, the housing 10, and the signal transmitter and receiver 11 are connected to each other so as to be movable forward and backward in the axial direction integrally with respect to the sheath 4 and the outer tube 5. Therefore, for example, when an operation of pushing the hub 8 toward the distal end side, that is, a push-in operation is performed, the inner tube 6 connected to the hub 8 is pushed into the outer tube 5 and the unit connector 7, and the drive shaft 9, the housing 10, and the signal transmitter and receiver 11, that is, the imaging core 12 moves forward inside the sheath 4, that is, toward the distal end side. For example, when an operation of pulling the hub 8 toward the proximal end side, that is, a pull-back operation is performed, the inner tube 6 is pulled out from the outer tube 5 and the unit connector 7 as indicated by an arrow A1 in FIGS. 1 and 2B, and the imaging core 12 moves toward the proximal end side inside the sheath 4 as indicated by an arrow A2.

As illustrated in FIG. 2A, when the inner tube 6 is pushed most toward the distal end side, a distal end of the inner tube 6 reaches the vicinity of a relay connector 13. At this time, the signal transmitter and receiver 11 is located at a distal end of the sheath 4 (near a distal end surface of a lumen of the sheath 4). The relay connector 13 connects the sheath 4 and the outer tube 5.

As illustrated in FIG. 2B, a locking portion 14 for preventing disengagement is provided at the distal end of the inner tube 6. The locking portion 14 prevents the inner tube 6 from coming out of the outer tube 5. The unit connector 7 includes a distal-end-side portion connector 7a and a proximal-end-side portion connector 7b detachably connected to the distal-end-side portion connector 7a. The locking portion 14 is configured to be hooked at a predetermined position on an inner wall of the proximal-end-side portion connector 7b of the unit connector 7 when the hub 8 is pulled to the maximum extent toward the proximal end side, that is, when the inner tube 6 is pulled out from the outer tube 5 and the unit connector 7 to the maximum extent. As the proximal-end-side portion connector 7b is detached from the distal-end-side portion connector 7a, the inner tube 6 including the locking portion 14 can be extracted from the outer tube 5.

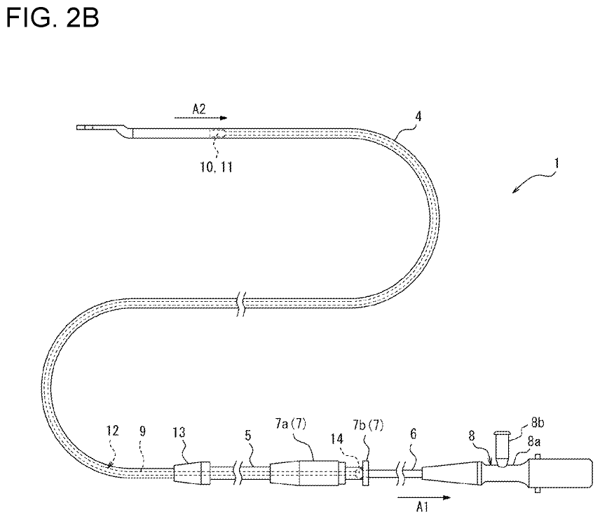

As illustrated in FIG. 3, the drive shaft 9 is an elongated hollow member, and an electric signal line (electric cable) 15 and an optical signal line (optical fiber) 16 connected to the signal transmitter and receiver 11 are disposed inside the drive shaft 9.

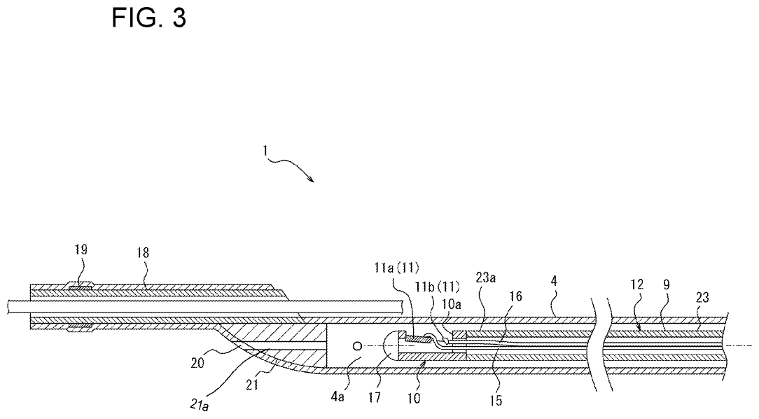

The signal transmitter and receiver 11 includes an ultrasound transmitter and receiver 11a that transmits and receives an ultrasound wave and an optical transmitter and receiver 11b that transmits and receives light. The ultrasound transmitter and receiver 11a includes a transducer that transmits an ultrasound wave based on a pulse signal into a body cavity and receives an ultrasound wave reflected from a biological tissue in the body cavity. The transducer is electrically connected to an electrical connector 15a (see FIG. 4) via the electric signal line 15. The transducer can be made of, for example, a piezoelectric material such as ceramics or quartz.

The optical transmitter and receiver 11b includes an optical element that transmits light into a body cavity and receives light reflected from a biological tissue in the body cavity. The optical element is optically connected to an optical connector 16a (see FIG. 4) via the optical signal line 16. The optical element can be formed using, for example, a lens such as a ball lens.

The signal transmitter and receiver 11 is accommodated in the housing 10. A proximal end of the housing 10 is fixed to the distal end of the drive shaft 9. The housing 10 is formed using a cylindrical tube made of metal, and is provided with an opening 10a on a peripheral surface thereof so as not to hinder the progress of a signal transmitted and received by the signal transmitter and receiver 11. The housing 10 can be formed by, for example, laser processing or the like. The housing 10 may also be formed by shaving from a metal lump, metallic powder injection molding (MIM), or the like.

A distal end member 17 is provided at a distal end of the housing 10. The distal end member 17 has a substantially hemispherical outer shape, and accordingly, suppresses friction and catching with an inner surface of the sheath 4. The distal end member 17 need not necessarily provided.

The sheath 4 has a lumen 4a into which the drive shaft 9 is inserted and in which the drive shaft 9 is positioned to be movable forward and backward. A tubular guide wire insertion member 18 through which a guide wire can pass is attached to the distal end of the sheath 4 and is positioned so that an axial center of the tubular guide wire insertion member 18 is shifted from an axial center of the lumen of the sheath 4. The sheath 4 and the guide wire insertion member 18 are integrated by thermal fusion or the like. The guide wire insertion member 18 is provided with a marker 19 having an X-ray contrast property. The marker 19 is made of a metal tube having high X-ray impermeability such as Pt or Au.

A communication hole 20 that communicates with the inside and the outside of the lumen 4a is formed at the distal end of the sheath 4. That is, the communication hole 20 communicates the lumen 4a inside the sheath 4 with the environment outside the sheath 4. In addition, a reinforcing member 21 joined to the guide wire insertion member 18 is provided at a distal end of the lumen 4a of the sheath 4. The reinforcing member 21 has a communication passage 21a formed to allow communication between the communication hole 20 and the inside of the lumen 4a disposed on the proximal end side of the reinforcing member 21. The reinforcing member 21 need not necessarily be provided at the distal end of the sheath 4.

The communication hole 20 is a priming solution discharge hole for discharging a priming solution. When the image diagnosis catheter 1 is used, the priming solution can be released from the communication hole 20 to the outside to discharge a gas such as air from the inside of the sheath 4 together with the priming solution at the time of performing a priming process of filling the inside of the sheath 4 with the priming solution.

A distal-end-side portion of the sheath 4, which is a range in which the signal transmitter and receiver 11 moves in the axial direction of the sheath 4, forms a window portion having a higher signal transmission property than other portions. The sheath 4, the guide wire insertion member 18, and the reinforcing member 21 are made of a flexible material, and the material is not particularly limited, examples thereof include various thermoplastic elastomers such as a styrene-based material, a polyolefin-based material, a polyurethane-based material, a polyester-based material, a polyimide-based material, a polyimide-based material, a polybutadiene-based material, a transpolyisoprene-based material, a fluororubber-based material, and a chlorinated polyethylene-based material, and one or a combination of two or more of these (a polymer alloy, a polymer blend, a laminate, or the like) can also be used.

As illustrated in FIG. 4, the hub 8 includes: a hub body 8a which has a tubular shape coaxial with the inner tube 6 and is integrally attached to the external device 2 in a detachable manner; a port 8b protruding radially outward from the hub body 8a and communicating with the inside of the hub body 8a; a connection tube 8c integrally attached to an outer peripheral surface of the drive shaft 9; a bearing 8d rotatably supporting the connection tube 8c; a seal member 8e that prevents the priming solution from leaking from a gap between the connection tube 8c and the bearing 8d toward the proximal end side; and a connector portion (connector) 8f provided with the electrical connector 15a and the optical connector 16a and integrally attached to a first drive unit 2a of the external device 2 in a detachable manner. The connector portion 8f is rotatable integrally with the connection tube 8c and the drive shaft 9.

The proximal end of the inner tube 6 is integrally connected to a distal end of the hub body 8a. The drive shaft 9 extends out from the inner tube 6 inside the hub body 8a.

As illustrated in FIG. 1, an injection device 22 (see FIG. 1) that injects the priming solution at the time of performing the priming process is connected to the port 8b. The injection device 22 includes a connector 22a connected to the port 8b and a syringe (not illustrated) connected to the connector 22a via a tube 22b.

The external device 2 includes the first drive unit 2a configured to rotationally drive the drive shaft 9 and a second drive unit 2b configured to move the drive shaft 9 in the axial direction (that is, for the push-in operation and the pull-back operation). The first drive unit 2a can be configured using, for example, an electric motor. The second drive unit 2b can be configured using, for example, an electric motor and a direct motion conversion mechanism. The direct motion conversion mechanism can convert rotational motion into linear motion, and can include, for example, a ball screw, a rack-and-pinion mechanism, or the like.

Operations of the first drive unit 2a and the second drive unit 2b are controlled by a control apparatus 2c electrically connected to the first drive unit 2a and the second drive unit 2b. The control apparatus 2c includes a central processing unit (CPU) and a memory. The control apparatus 2c is electrically connected to a display 2d.

A signal received by the ultrasound transmitter and receiver 11a is transmitted to the control apparatus 2c via the electrical connector 15a, subjected to predetermined processing, and displayed as an image on the display 2d. A signal received by the optical transmitter and receiver 11b is transmitted to the control apparatus 2c via the optical connector 16a, subjected to predetermined processing, and displayed as an image on the display 2d.

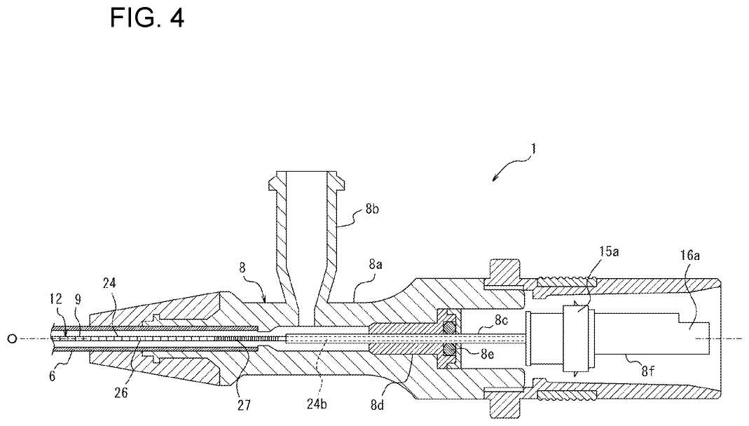

As illustrated in FIG. 5, the drive shaft 9 includes: a coil shaft 23 having a distal end 23a (see FIG. 3) fixed to the proximal end of the housing 10; and a shaft member 24 having a distal end 24a fixed to a proximal end 23b of the coil shaft 23 and having higher torsional stiffness than torsional stiffness of the coil shaft 23. An axial length of the drive shaft 9 is preferably 1200 mm or more and 2000 mm or less. An outer diameter of the drive shaft 9 is not particularly limited, and is, for example, 0.56 mm. An inner diameter of the drive shaft 9 is not particularly limited, and is, for example, 0.3 mm.

The coil shaft 23 can be formed using, for example, multiple coils 23c positioned in a multi-layer arrangement. The coils 23c may have different winding directions. In the embodiment shown in FIG. 5, the coils 23c axially overlap one another and are positioned inside one another. Each of the coils 23c is usually of a multi-wire winding type. That is, each of the coils 23c may be formed by multiple wires that are wound together.] Although the coil shaft 23 is formed using the three-layer coils 23c of a double winding type in the example illustrated in FIG. 5, the number of layers and the number of wires can be appropriately changed. Each of the coils 23c is made of metal such as stainless steel or a nickel-titanium (Ni—Ti) alloy, for example. A diameter (outer diameter) of a peripheral surface of the proximal end 23b of the coil shaft 23 is reduced by cutting.

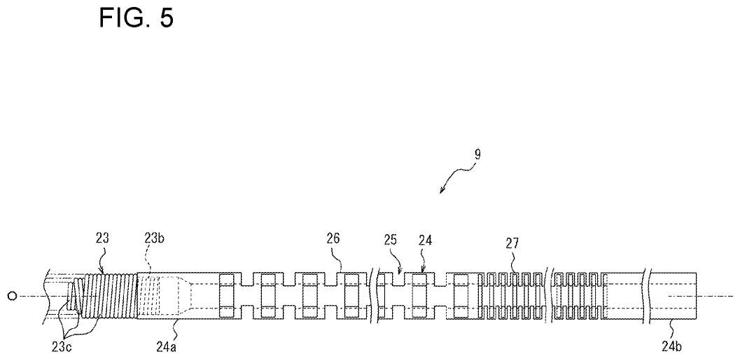

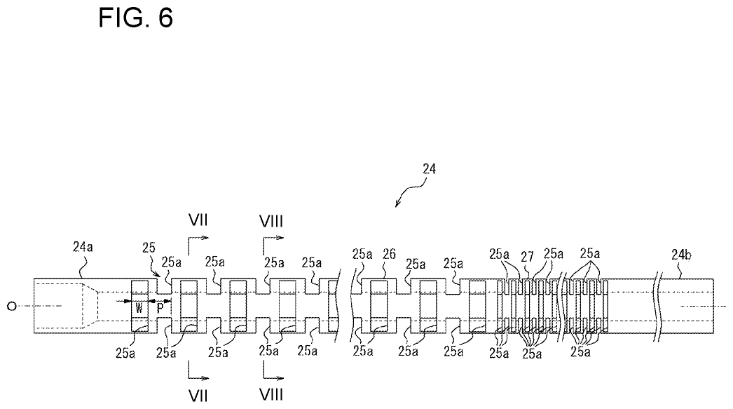

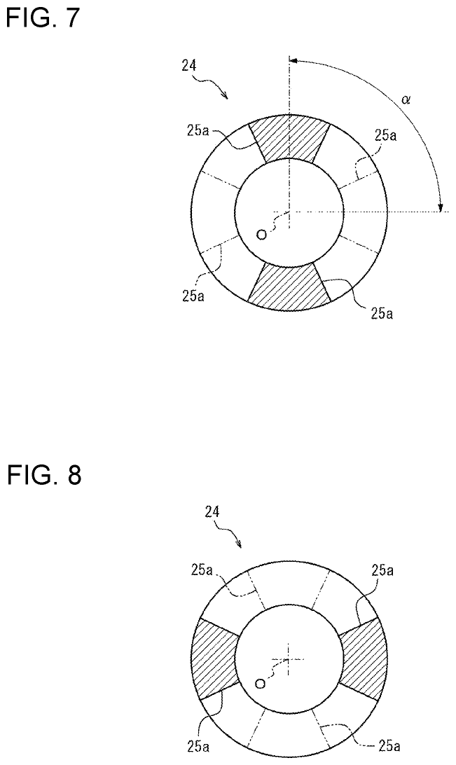

As illustrated in FIGS. 6 to 8, the shaft member 24 is a tube or tubular member made of metal such as stainless steel or a nickel-titanium (Ni—Ti) alloy having a notch 25. The notch 25 includes a plurality of slits 25a.

The shaft member 24 includes: a main portion 26 in which a plurality of slits 25a each having a predetermined width W and arranged in the circumferential direction are disposed in a predetermined pattern where the slits 25a are arranged side by side at a predetermined pitch P in the axial direction; and a weakened portion 27 that has lower torsional strength, that is, is more easily twisted and ruptured (broken), than the torsional strength of each of the main portion 26 and the coil shaft 23. The weakened portion 27 is located closer to the proximal end side than the main portion 26. That is, the weakened portion 27 is proximal of the main portion 26. The main portion 26 occupies most of an axial length of the shaft member 24. The axial length of the main portion 26 is greater than the axial length of the weakened portion 27. An axial length of the weakened portion 27 is preferably 10 mm or more and 50 mm or less, and more preferably 10 mm or more and 30 mm or less.

The distal end 24a of the shaft member 24 is located distally beyond the distal end of the main portion 26, and the proximal 24b end of the shaft member 24 is located proximally beyond the proximal end of the weakened portion 27. Thus, as shown in FIG. 5, the distal portion of the shaft member 24 is devoid of the slits that are located in the main portion 26, and the proximal portion of the shaft member 24 is devoid of the slits that are located in the weakened portion 27. An inner peripheral surface of the distal end 24a of the shaft member 24 is enlarged in diameter at a distal-end-side portion by cutting. That is, as shown in FIG. 6, the inner diameter of the distal end 24a of the shaft member 24 is enlarged relative to the inner diameter of the portion of the shaft member 24 that is axially adjacent the enlarged inner diameter part. As illustrated in FIG. 5, the proximal end 23b of the coil shaft 23 is inserted into or positioned in the distal end 24a of the shaft member 24, and is fixed by welding using, for example, solder. As illustrated in FIG. 4, the connection tube 8c is integrally attached to the proximal end 24b of the shaft member 24.

The predetermined pattern of slits mentioned above regarding the main portion 26 is a pattern in which pairs of the slits 25a, each of the pairs of slits 25a facing each other in the radial direction and each having the predetermined width W and extending along the circumferential direction, are disposed so as to be arranged side by side at the predetermined pitch P in the axial direction while rotating by a predetermined angle α. Thus, as shown in FIGS. 5-8, two slits 25a forming one pair of slits are positioned at one axial location on the main portion 26, with the two slits 25a forming the one pair each having the predetermined width W and being arranged radially opposite one another, while two other slits 25a forming another pair of slits are positioned at an axially adjacent location on the main portion 26, with the two slits 25a forming the other pair each having the predetermined width W and being arranged radially opposite one another. In addition, the two slits 25a forming the one pair of slits are spaced by the predetermined pitch P from the two slits 25a forming the other pair of slits, and the two slits 25a forming the one pair of slits are circumferentially shifted by the predetermined angle α. This arrangement of axially spaced pairs of the slits 25a continues along the length of the main portion as shown in FIGS. 5 and 6. In the example illustrated in FIGS. 5 to 8, the predetermined angle α is 90°. The predetermined angle α is not limited to 90°.

In addition, the predetermined pattern may be a pattern in which pairs of the slits 25a, each of the pairs of the slits 25a facing each other in the radial direction and each having the predetermined width W and extending obliquely with respect to the circumferential direction, are disposed to be arranged side by side at the predetermined pitch P in the axial direction while being circumferentially shifted by the predetermined angle α. The predetermined pattern may be a pattern in which three or more slits 25a each having the predetermined width W and arranged in the circumferential direction are arranged side by side at the predetermined pitch P in the axial direction. That is, instead of arranging pairs of the slits in the manner discussed above, three of the slits may be arranged in the manner discussed above. Also, FIGS. 5-8 show that each of the slits extends over only a portion of the circumferential extent of the main portion 26 of the tubular shaft member 24. That is, each of the slits extends over less than an entirety of the circumferential extent of the main portion 26 of the tubular shaft member 24.

In the weakened portion 27, the plurality of slits 25a are disposed in a pattern identical to the pattern of the main portion 26 except that a predetermined width W and a predetermined pitch P are smaller than those of the main portion 26. The weakened portion 27 may have a configuration in which only one of the predetermined width W and the predetermined pitch P of the plurality of slits 25a is smaller than that of the main portion 26.

Each of the slits 25a in the main portion 26 and the weakened portion 27 can be formed by, for example, cutting with a laser beam that is scanned in the circumferential direction while passing through the central axis O of the shaft member 24.

The predetermined width W, the predetermined pitch P, and a circumferential length of the slit 25a in the main portion 26 can be appropriately set. The predetermined width W of the slit 25a in the main portion 26 is, for example, 0.15 mm. The predetermined pitch P of the slits 25a in the main portion 26 is, for example, 0.25 mm. The circumferential length of the slit 25a in the main portion 26 is, for example, 0.63 mm (the length on an outer peripheral surface of the main portion 26).

The predetermined width W, the predetermined pitch P, and a circumferential length of the slit 25a in the weakened portion 27 can be appropriately set. The predetermined width W of the slit 25a in the weakened portion 27 is, for example, 0.02 mm. The predetermined pitch P of the slits 25a in the weakened portion 27 is, for example, 0.07 mm. The circumferential length of the slit 25a in the weakened portion 27 is, for example, 0.63 mm (the length on an outer peripheral surface of the weakened portion 27).

As illustrated in FIG. 4, the weakened portion 27 is provided at the proximal end of the drive shaft 9 and is located inside the hub 8. Therefore, the weakened portion 27 is located closer to the proximal end side than the sheath 4 when the inner tube 6 advances most, and is located closer to the proximal end side than the unit connector 7 when the inner tube 6 advances most. That is, the weakened portion 27 is positioned proximal of the proximal end of the sheath 4 when the inner tube 6 advances most, and is located proximal of the proximal end of the unit connector 7 when the inner tube 6 advances most.

At the time of diagnosis, the imaging core 12 retracts at a constant speed inside the lumen 4a of the sheath 4 by the pull-back operation by the second drive unit 2b of the external device 2 in a state where the sheath 4 is inserted into (positioned in) a body cavity and the imaging core 12 is rotationally driven at a constant rotational speed of about 1000 to 10000 rpm by the first drive unit 2a of the external device 2. At this time, the control apparatus 2c of the external device 2 causes the signal transmitter and receiver 11 to transmit and receive a signal. A state of a tissue around the body cavity is displayed as an image on the display 2d based on the signal received by scanning performed by the rotation and the retraction of the imaging core 12.

At the time of such signal scanning, when the imaging core 12 comes into contact with the sheath 4 bent due to a flexure or a lesion of a biological lumen and the drive shaft 9 resonates due to the contact, image distortion called NURD occurs.

In the present embodiment, however, the drive shaft 9 includes not only the coil shaft 23 but also the shaft member 24 having higher torsional stiffness than the torsional stiffness of the coil shaft 23, and thus, the natural frequency of the drive shaft 9 can be increased. Therefore, a rotational speed region where the drive shaft 9 resonates is made greater than an upper limit (for example, 10,000 rpm) of an applicable rotational speed of the imaging core 12 that can be set by the external device 2, whereby the occurrence of NURD can be suppressed. In a case where the applicable rotational speed of the imaging core 12 can be selected from among a plurality of stages by the external device 2, the rotational speed region where the drive shaft 9 resonates may be adjusted to a size deviating from any applicable rotational speed that is selectable (for example, adjusted to a size just around the middle between 3600 rpm and 5600 rpm, for example, in a case where three of 1800 rpm, 3600 rpm, and 5600 rpm are selectable), thereby suppressing the occurrence of NURD.

In addition, a distal-end-side portion of the drive shaft 9 is configured using the coil shaft 23 in the present embodiment, and thus, flexibility and kink resistance can be easily secured in the distal-end-side portion, thereby enabling stable signal scanning. In addition, a proximal-end-side portion of the drive shaft 9 is configured using the shaft member 24 in the present embodiment, and thus, buckling resistance can be easily secured in the proximal-end-side portion, thereby enabling an easy push-in operation.

Here, an axial length of the coil shaft 23 is preferably 250 mm or more and 1000 mm or less. In the case of being 250 mm or more, the drive shaft 9 can flexibly follow a flexed biological lumen and perform scanning, and the stable signal scanning can be more reliably performed. In the case of being 1000 mm or less, the occurrence of NURD can be more reliably suppressed.

In addition, the axial length of the shaft member 24 is preferably 200 mm or more and 1750 mm or less. In the case of being 200 mm or more, the occurrence of NURD can be more reliably suppressed, and the push-in operation can be more reliably and easily performed. In the case of being 1750 mm or less, the stable signal scanning can be more reliably performed by sufficiently securing the axial length of the coil shaft 23.

In general, if the imaging core 12 continues to rotate in a state where the housing 10 or the like is in contact with the sheath 4 in a case where the sheath 4 is inserted into a narrow lesion portion, a sharply curved vessel, or the like, the sheath 4 may be damaged by friction between the sheath 4 and the housing 10 or the like.

In the present embodiment, however, the shaft member 24 includes the weakened portion 27 having lower torsional strength than the torsional strength of each of the main portion 26 and the coil shaft 23. That is, the drive shaft 9 has the weakened portion 27 in which the torsional strength is locally reduced to be lower than any other portion of the drive shaft 9. Therefore, when the imaging core 12 continues to rotate in a state where the housing 10 or a portion of the drive shaft 9 on the distal end side of the weakened portion 27 is in contact with the sheath 4, the weakened portion 27 is first twisted and ruptured (broken), so that the rotation of the imaging core 12 on the distal end side of the weakened portion 27 can be stopped, thereby suppressing the damage to the sheath 4.

In addition, since the weakened portion 27 is located closer to the proximal end side than the sheath 4 (i.e., the weakened portion 27 is proximal of the sheath 4) when the inner tube 6 advances most in the present embodiment, it is possible to suppress the sheath 4 from being damaged due to contact with a sharp cut surface of the weakened portion 27 when the weakened portion 27 is twisted and cut.

Since the weakened portion 27 is located closer to the proximal end side than the unit connector 7 (i.e., the weakened portion 27 is proximal of the unit connector 7) when the inner tube 6 advances most in the present embodiment, a distal-end-side portion of the cut weakened portion 27 can be exposed to the outside by releasing holding of the inner tube 6 performed by the unit connector 7 after the weakened portion 27 has been twisted and cut, and the imaging core 12 can be easily removed from the inside of the sheath 4 by gripping the exposed weakened portion 27.

Since the weakened portion 27 is provided at the proximal end of the drive shaft 9 in the present embodiment, the damage to the sheath 4 can be more reliably suppressed.

Since the weakened portion 27 is located closer to the proximal end side than the main portion 26 in the present embodiment (i.e., the weakened portion 27 is proximal of the main portion 26), the damage to the sheath 4 can be more reliably suppressed from this point as well.

Since the shaft member 24 is formed using the tube having the notch 25 in the present embodiment, it is possible to achieve the shaft member 24 having both appropriate flexibility and appropriate torsional stiffness for easy bending and deformation.

Since the notch 25 has a non-spiral shape in the present embodiment, appropriate torsional stiffness can be easily achieved.

Since the notch 25 includes the plurality of slits 25a extending along the circumferential direction in the present embodiment, the shaft member 24 having both appropriate flexibility and appropriate torsional stiffness can be easily achieved.

Since the shaft member 24 has the main portion 26 in which the plurality of slits 25a each having the predetermined width W and arranged in the circumferential direction are disposed in the predetermined pattern where the slits 25a are arranged side by side at the predetermined pitch P in the axial direction in the present embodiment, it is possible to achieve the shaft member 24 having both high flexibility and high torsional stiffness.

Since the predetermined pattern is a pattern in which the pairs of slits 25a, each of the pairs of slits 25a facing each other in the radial direction and each having the predetermined width W and extending along the circumferential direction, are disposed so as to be arranged side by side at the predetermined pitch P in the axial direction while rotating by the predetermined angle α in the present embodiment, the shaft member 24 having both high flexibility and high torsional stiffness can be more reliably achieved.

Since the plurality of slits 25a are disposed in the weakened portion 27 in a pattern identical to the pattern of the main portion 26 except that the predetermined width W and the predetermined pitch P are smaller than those of the main portion 26 in the present embodiment, the weakened portion 27 can be formed only by changing the predetermined width W and the predetermined pitch P, whereby the shaft member 24 can be easily formed.

The above-described embodiment is merely an example of the present disclosure, and various modifications such as those described below can be made.

The drive shaft 9 is the drive shaft 9 for constituting the imaging core 12 of the image diagnosis catheter 1, and can be variously modified as long as the coil shaft 23 having the distal end 23a fixed to the proximal end of the housing 10 that accommodates the signal transmitter and receiver 11, and the shaft member 24 fixed to the proximal end 23b of the coil shaft 23 and having higher torsional stiffness than torsional stiffness of the coil shaft 23 are provided.

By way of example, the axial length of the coil shaft 23 is preferably 250 mm or more and 1000 mm or less.

The axial length of the shaft member 24 is preferably 200 mm or more and 1750 mm or less.

The shaft member 24 is preferably a tube having the notch 25.

The notch 25 preferably has a non-spiral shape.

The notch 25 preferably includes the plurality of slits 25a extending along the circumferential direction.

The shaft member 24 preferably has the main portion 26 in which the plurality of slits 25a each having the predetermined width W and arranged in the circumferential direction are arranged in a predetermined pattern to be arranged side by side at the predetermined pitch P in the axial direction.

The shaft member 24 preferably includes the weakened portion 27 having lower torsional strength than torsional strength of each of the main portion 26 and the coil shaft 23.

In the weakened portion 27, the plurality of slits 25a are preferably disposed in a pattern identical to the pattern of the main portion 26 except that the predetermined width W and/or the predetermined pitch P are smaller than those of the main portion 26.

The weakened portion 27 is preferably located closer to the proximal end side than the main portion 26.

The predetermined pattern is preferably a pattern in which pairs of the slits 25a, each of the pairs of the slits 25a facing each other in the radial direction and each having the predetermined width W and extending along the circumferential direction, are disposed so as to be arranged side by side at the predetermined pitch P in the axial direction while rotating by the predetermined angle α.

The image diagnosis catheter 1 can be variously modified as long as the imaging core 12, which includes the drive shaft 9, the housing 10 fixed to the distal end 23a of the coil shaft 23, and the signal transmitter and receiver 11 accommodated in the housing 10, and the sheath 4 into which the imaging core 12 is inserted are provided.

The image diagnosis catheter 1 preferably includes the outer tube 5 connected to the proximal end of the sheath 4 and the inner tube 6 inserted into the outer tube 5 so as to be movable forward and backward integrally with the imaging core 12.

The weakened portion 27 is preferably located closer to the proximal end side than the sheath 4 when the inner tube 6 advances most.

It is preferable that the image diagnosis catheter 1 include the unit connector 7 that is connected to the proximal end of the outer tube 5, holds the inner tube 6 so as to be movable forward and backward, and can release the holding of the inner tube 6, and that the weakened portion 27 be located closer to the proximal end side than the unit connector 7 when the inner tube 6 advances most.

The weakened portion 27 is preferably located at the proximal end of the drive shaft 9.

The detailed description above describes embodiments of a drive shaft and an image diagnosis catheter representing examples of the new drive shaft and image diagnosis catheter disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. An image core drive shaft that is to be used in an image diagnosis catheter, the image core drive shaft comprising:
a coil shaft having a distal end configured to be fixed to a proximal end of a housing that accommodates a signal transmitter and receiver, the coil shaft possessing a proximal end and a distal end, the coil shaft including at least one wire that is wound into a coil; and
a shaft member fixed to the proximal end of the coil shaft and having higher torsional stiffness than torsional stiffness of the coil shaft.

2. The image core drive shaft according to claim 1, wherein an axial length of the coil shaft is 250 mm or more and 1000 mm or less.

3. The image core drive shaft according to claim 1, wherein an axial length of the shaft member is 200 mm or more and 1750 mm or less.

4. The image core drive shaft according to claim 1, wherein the shaft member is a tubular member that possesses an outer surface provided with a notch.

5. The image core drive shaft according to claim 4, wherein the notch has a non-spiral shape.

6. The image core drive shaft according to claim 4, wherein the notch includes a plurality of pairs of slits, the pairs of slits being axially spaced apart along a length of the tube, each of the slits extending along a circumferential direction.

7. The image core drive shaft according to claim 4, wherein the notch includes a plurality of slits each of which extends along less than entirety of a circumferential extent of the tubular member.

8. The image core drive shaft according to claim 1, wherein the shaft member includes a longitudinally extending main portion in which a plurality of slits each having a width and arranged in a circumferential direction of the main portion are disposed in a pattern, the slits being axially spaced apart at a pitch along a longitudinal extent of the main portion.

9. The image core drive shaft according to claim 8, wherein the shaft member includes a weakened portion having lower torsional strength than a torsional strength of each of the main portion and the coil shaft.

10. The image core drive shaft according to claim 9, wherein a plurality of slits are disposed in the weakened portion in a pattern identical to the pattern of the slits in the main portion, except that a width of the slits in the weakened portion is smaller than the width of the slits in the main portion and/or a pitch of the slits in the weakened portion is smaller than the pitch of the slits in the main portion.

11. The image core drive shaft according to claim 8, wherein the weakened portion is proximal of the main portion.

12. The image core drive shaft according to claim 8, wherein the pattern of slits in the longitudinally extending main portion is a pattern in which the slits are arranged in pairs, the pairs of slits being arranged side by side at the pitch in the axial direction while being circumferentially shifted by a predetermined angle, the slits in each pair facing each other in a radial direction.

13. An image diagnosis catheter comprising:
an imaging core comprised of an image core drive shaft according to claim 1; and
a sheath in which the imaging core is positioned.

14. An image diagnosis catheter comprising:
an imaging core and a tubular sheath in which the imaging core is positioned, the imaging core comprising:
a drive shaft, a housing, and a signal transmitter and receiver positioned in the housing and connected to a signal line extending inside the drive shaft,
the drive shaft comprising: a tubular shaft member and a tubular coil shaft; the tubular coil shaft possessing a distal end to which the housing is connected, the tubular shaft member possessing a distal end and a proximal end, the distal end of the tubular shaft member being connected to the tubular coil shaft, the coil shaft including at least one wire that is wound into a coil, the tubular shaft member being comprised of a main portion that extends from the distal end of the tubular shaft toward the proximal end of the tubular shaft member, the tubular shaft member including a plurality of slits that are axially spaced apart from one another, the main portion of the tubular shaft member at which are located the plurality of slits having a higher torsional stiffness than a torsional stiffness of the coil shaft.

15. The image diagnosis catheter according to claim 14, wherein the slits are arranged in pairs, the slits of each pair facing each other in a radial direction.

16. The image diagnosis catheter according to claim 14, wherein the main portion of the tubular shaft member possesses a circumference, each of the slits extending along only a portion of the circumference of the main portion of the tubular shaft member.

17. The image diagnosis catheter according to claim 14, wherein axially adjacent slits are offset from each other in a circumferential direction of the main portion of the tubular shaft member.

18. The image diagnosis catheter according to claim 17, wherein the slits are arranged in pairs so that two slits forming each pair are positioned at a common axial position along the main portion of the tubular shaft member, the axially adjacent pairs of the slits being offset from each other in the circumferential direction of the main portion of the tubular shaft member.

19. The image diagnosis catheter according to claim 14, wherein the tubular shaft member includes a weakened portion positioned proximal of the main portion of the shaft member, the weakened portion including a plurality of slits, the weakened portion having a lower torsional strength than a torsional strength of the main portion and a torsional strength of the coil shaft.

20. A method comprising:
positioning an imaging core, located in a lumen of a surrounding sheath, inside a body cavity in a living body, the imaging core comprising a rotatable and axially movable drive shaft, the imaging core also comprising a housing in which is located a signal transmitter and receiver, the drive shaft being comprised of a coil shaft fixed to a distal end of a shaft member so that the coil shaft and the shaft member rotate and axially move together, the coil shaft having a distal end that is fixed to the housing so that rotation and axial movement of the drive shaft results in rotation and axial movement of the housing as well as the signal transmitter and receiver accommodated in the housing, the coil shaft including at least one wire that is wound into a coil, the shaft member having a higher torsional stiffness than a torsional stiffness of the coil shaft; and
retracting the imaging core in the lumen of the surrounding sheath and rotating the imaging core in the lumen of the surrounding sheath while the signal transmitter and receiver transmits and receives signals.
